(19) United States
(12) Patent Application Publication
(10) Pub. No.: US 2017/0252312 A1
(43) Pub. Date: Sep. 7, 2017

(54) PREPARATIONS FOR TREATMENT AND PREVENTION OF ALCOHOL FLUSHING AND ALCOHOL-INDUCED HYPERSENSITIVITY REACTIONS

(71) Applicant: BIOHIT OYJ, Helsinki (FI)

(72) Inventors: Semi KORPELA, Helsinki (FI); Kari SYRJANEN, Helsinki (FI); Mikko SALASPURO, Helsinki (FI); Osmo SUOVANIEMI, Helsinki (FI)

(73) Assignee: BIOHIT OYJ, Helsinki (FI)

(21) Appl. No.: 15/511,126

(22) PCT Filed: Sep. 15, 2015
(86) PCT No.: PCT/FI2015/050612

§ 371 Date: Mar. 14, 2017

(30) Foreign Application Priority Data
Sep. 15, 2014 (FI) 20145806

(51) Int. Cl. A61K 31/198; A61K 9/48; A61K 9/00; A61K 9/20

(52) U.S. Cl. CPC A61K 31/198; A61K 9/20; A61K 9/48; A61K 9/0053; A61K 9/0056

(57) ABSTRACT

The present invention relates to a cysteine releasing pharmaceutical preparation(s) for novel use in preventing and/or treating alcohol flushing and alcohol induced hypersensitivity reactions. Additionally, the invention relates to a method for eliminating histamine releasing effect of locally formed acetaldehyde in the digestive tract.

### TECHNICAL FIELD

The present invention relates to the prevention and/or treatment of effects associated with alcohol consumption and more specifically to methods and compositions for the prevention or treatment of alcohol-induced flushing and hypersensitivity reactions.

### BACKGROUND

Alcohol flush reaction (also known as Asian flush syndrome, Asian flush, Asian glow, among others) is a condition in which an individual develops flushes or blotches associated with erythema on the face, neck, shoulders, and, in some cases, the entire body after consuming alcoholic beverages. The reaction is the result of an accumulation of acetaldehyde, a metabolic byproduct of the catabolic metabolism of alcohol, and is caused by an aldehyde dehydrogenase deficiency.

Based on strong scientific evidence this syndrome has been associated with an increased risk of oral, pharyngeal, esophageal and gastric cancer in those who drink alcohol. It has also been associated with lower than average rates of alcoholism, possibly due to its association with adverse effects after drinking alcohol.

Metabolism of alcohol takes place mainly in the liver and involves a two-step enzymatic reaction. First, alcohol is oxidized to acetaldehyde by cytoplasmic alcohol dehydrogenase (ADH) enzyme. Secondly, acetaldehyde is oxidized to acetic acid by mitochondrial and cytoplasmic aldehyde dehydrogenase (ALDH) enzymes. Acetic acid is then transported from the liver to the muscles and adipose tissue where it is further broken down into carbon dioxide and water. Thus, acetaldehyde concentrations in blood and tissues are regulated by balance of both alcohol—and ALDH enzymes. This is important, because although ethanol is toxic to the body, acetaldehyde is much more toxic than ethanol. Furthermore, acetaldehyde derived from alcoholic beverages has been classified as a Group 1 carcinogen for humans by the International Agency for Research on Cancer (IARC/WHO)(IARC 2012).

In the majority of humans, the capacity of liver to eliminate acetaldehyde formed from ethanol is so efficient that measurable levels of acetaldehyde do not escape from the liver to blood circulation. However, the situation may be different in other tissues. For instance, in the digestive tract both microbes and mucosal cells are able to produce locally acetaldehyde from ethanol, but are not able to eliminate it. Therefore, in the presence of ethanol, significant amounts of acetaldehyde accumulate in the oral cavity, esophagus, stomach and large intestine (Salaspuro 2003, 2009, 2011). In fact, the highest levels of ethanol-derived acetaldehyde are found in the digestive tract.

Approximately 36% of East Asians (Japanese, Chinese, Taiwanese and Koreans) show a characteristic physiological response to drinking alcohol that include facial flushing, increased skin temperature, tachycardia, decrease of diastolic blood pressure and sometimes also nausea (Kupari et al. 1983, Väkeväinen et al. 2001, Brooks et al. 2009). This so-called alcohol flushing response (also known as “Asian flush” or “Asian glow”) is predominantly due to an inherited deficiency of the main enzyme for acetaldehyde oxidation, mitochondrial aldehyde dehydrogenase 2 (ALDH2). It has been estimated that there are at least 540 million ALDH2-deficient individuals in the world representing approximately 8% of the global population.

In East Asians (Orientals), the mechanism of Asian flush and hypersensitivity reactions e.g. bronchoconstriction in human asthmatics, has been proposed to be elevated blood levels of acetaldehyde, secondary to decreased elimination by deficient ALDH2-enzyme. Acetaldehyde has been shown to enhance histamine release from human and rat mast cells (Shimoda et al. 1996, Koivisto et al. 1999). The same mechanism may be involved also in the pathogenesis of allergic rhinitis (hay fever), allergic conjunctivitis, atopic dermatitis, eosinophilic esophagitis, anaphylaxis, chronic bronchitis, and chronic obstructive pulmonary disease (COPD) (Nihlen et al. 2005, Linneberg et al. 2008). This concept is strongly supported by the observations that histamine receptor antagonists (H2 blockers) and drugs inhibiting histamine release do alleviate alcohol-related hypersensitivity reactions (Miller et al. 1988, Myou et al. 1995, Takao et al. 1999). Furthermore, combined use of H₁₋ and H₂ receptor antagonists has been shown to neutralize alcohol-induced cutaneous flushing and systolic hypotension in Orientals (Miller et al. 1998).

More recently, alcohol-related and genetically determined hypersensitivity reactions have been described also in Caucasian populations (Linneberg et al. 2008). Indeed, 14% of the general adult population in Denmark had experienced hypersensitivity symptoms from the nose, lungs, or skin following intake of alcoholic drinks. These reactions were proposed to be caused by a mechanism similar to that described in East Asians, i.e., histamine-releasing effect of acetaldehyde (Linneberg et al. 2008). These alcohol hypersensitivity reactions were found to be associated with a genetically determined fast metabolism of ethanol i.e., enhanced production of acetaldehyde—the A allele of ADH1b and ALDH1b1, the function of which is not yet known (Linneberg et al. 2010).

In subjects with normal ALDH2-enzyme, alcohol drinking does not result in detectable levels of free acetaldehyde in peripheral blood (Lindroos et al. 1980). However, after a small or moderate dose of alcohol, markedly high acetaldehyde levels are found in the saliva (Homann et al. 1997, Väkeväinen et al. 2000). This is by and large due to oral microbes and mucosal cells that possess significant ADH activity metabolizing ethanol to acetaldehyde locally (Homann et al. 1997, Dong et al. 1996), but which are not able to eliminate it (Salaspuro 2003, 2009).

In contrast to Caucasians, slightly elevated blood acetaldehyde levels after alcohol intake have been demonstrated among ALDH2-deficient East-Asians, and this has been suggested to provide the basis for their flushing, hypersensitivity reactions and cardiovascular responses seen after alcohol intake (Mizoi et al. 1979, Chen et al. 2009). However, in ALDH2-deficient individuals, salivary acetaldehyde levels after a single dose of alcohol are 10-20 times higher than those in the blood, and 2-3 times higher than in individuals with normal ALDH2-enzyme (Väkeväinen et al. 2000, Yokoyama et al. 2008). Furthermore, after intragastric infusion of alcohol, ALDH2- deficient subjects have 5-6 times higher acetaldehyde levels in their gastric juice than those with normal ALDH2-enzyme.

The significant effects of local acetaldehyde in the G-I tract and consequently in the pathogenesis of flushing and hypersensitivity reactions is supported also by other evidence. Accordingly, in ALDH2-deficient individuals, a potent inhibitor of ADH enzyme (4-methylpyrazole, 4-MP) has been shown to markedly decrease alcohol-induced increase in salivary acetaldehyde levels (Väkeväinen et al. 2001). In these persons, 4-MP suppressed also significantly the flushing response to alcohol. Similarly, the increase in heart rate and skin temperature as well as the drop in diastolic blood pressure disappeared when such persons used 4-MP before ethanol intake (Väkeväinen et al. 2001).

Based on the above discussed observations, it is plausible that the acetaldehyde-mediated flushing and hypersensitivity reactions after alcohol intake are not caused by elevated blood acetaldehyde levels. In contrast, local acetaldehyde-mediated release of histamine from the tissue mast cells in the upper digestive tract mucosa could be the major mechanism explaining these reactions. It is a common knowledge that upper G-I-tract contains abundantly histamine releasing mast cells (Syrjänen 1975, Steer 1976, Walsh 2003).

WO 2012/027603 relates to therapeutic consumable compositions for reducing facial flush effect incident to alcohol consumption in some persons of Asian descent or persons with ALDH2 gene deficiency. The composition is a single dose and designed for lowering blood alcohol level and removing acetaldehyde from the liver.

WO 2014/011676 and US 2014/256760 are directed to compositions comprising for example N-acetylcysteine for reducing alcohol reaction and relating unpleasant symptoms that accompany the consumption of alcoholic beverages in a subject. Both applications are, however, silent about histamine-releasing effect of locally formed acetaldehyde.

### SUMMARY OF THE INVENTION

The present invention relates to cysteine-releasing pharmaceutical preparations for use in preventing and/or treating deleterious effects associated with alcohol consumption (i.e. in harm reduction). The preparations include for example slowly L-cysteine releasing preparations either in the mouth or stomach.

More precisely, the use according to the present invention is mainly characterized in that, what is stated in the characterizing part of claim 1. Additionally, the method according to the present invention is mainly characterized in claim 14.

One major advantage of the present invention is that alcohol-induced flushing and hypersensitivity reactions can be markedly prevented and treated by eliminating and inactivating acetaldehyde by using preparation that release cysteine in the stomach or in oral cavity, as herein disclosed.

Next, the invention is described by the following figures and embodiments.

### SHORT DESCRIPTION OF FIGURES

FIG. 1: Chemical formula illustrating the covalent binding of acetaldehyde to L-cysteine. As a result, an inactive stable product, 2-methylthiazolidine-4-carboxylic acid (MTCA) is formed.

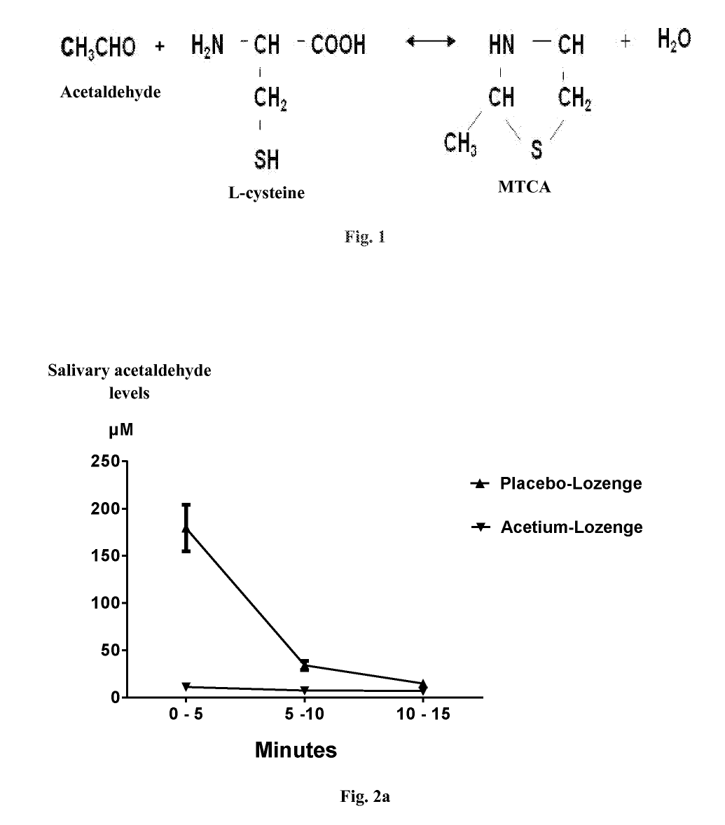

FIGS. 2a and 2b: The effect of slowly L-cysteine releasing Acetium-Lozenge on acetaldehyde levels in saliva (a) and areas under the curve (b)(means±SEM). Four volunteers sucked one Acetium-Lozenge containing either 3 mg L-cysteine or placebo. Thereafter they rinsed their mouths with 5 ml of Grappa (Grappa Di Monovitigino, acetaldehyde 5344 μM, alcohol 35.6 vol %) for 5 seconds. Grappa was spitted off and volunteers took another Acetium-Lozenge and sucked it for the rest of the experiment.

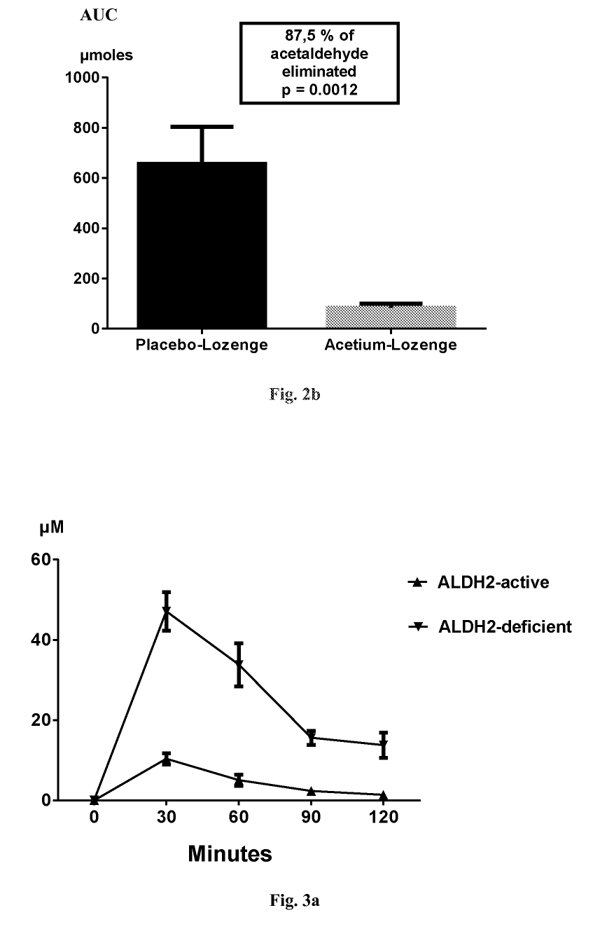

Thereafter whole saliva was collected as pooled samples for following 0-5, 5-10 and 10-15 minutes and their acetaldehyde and ethanol concentrations were determined by head space gas chromatography.

FIGS. 3a and 3b: The effect of ALDH2-deficiency on gastric juice acetaldehyde levels (a) and areas under the curve (b) after intragastric infusion of ethanol (15 vol %, 0.5 g/kg) (means±SEM). 10 ALDH2-active and 10 ALDH2-negative volunteers participated in the study.

FIGS. 4a and 4b: Effect of slowly L-cysteine releasing capsule (200 mg) in PPI treated ALDH2-deficient individuals on gastric juice acetaldehyde levels (a) and areas under the curve (b) after intragastric infusion of ethanol (15 vol %, 0.5 g/kg) (means±SEM).

### DESCRIPTION OF EMBODIMENTS

Based on prior art, alcohol-induced flushing and hypersensitivity reactions have been assumed to be caused by elevated levels of blood acetaldehyde resulting in enhanced release of histamine from the mast cells. However, after alcohol drinking, significantly higher acetaldehyde levels have been documented in saliva and gastric juice than in blood.

Based on well documented experimentation and other pertinent data, the inventors of the present invention have reached the conclusion that acetaldehyde-mediated release of histamine from the tissue mast cells found in abundance in the upper digestive tract mucosa is the most plausible mechanism behind alcohol-induced flushing and hypersensitivity reactions.

According to the disclosure, the present invention thus relates to treating and preventing of alcohol flushing and alcohol-induced hypersensitivity reactions by acetaldehyde-binding pharmaceutical preparations.

Cysteine and its derivatives are especially well suited to the purpose of the present invention. The most suitable amino acids for the use according to the invention are L- and D-cysteine. Other suitable amino acids or other compounds that avidly bind acetaldehyde and comprise a free sulphydryl (SH) and/or (NH₂) group include e.g.

Cysteic acid,

Cysteine glycine,

Threo or erythro-62 -phenyl-DL-cysteine,

β-tetramethylene-DL-cysteine,

D-penicillamine,

D,L-homocysteine,

N-acetylcysteine,

L-cysteinyl-L-valine,

β-β-tetramethylene-DL-cysteine, and

Erythro-beta-phenyl-DL-cysteine

However, only such acetaldehyde-binding compounds that cause no health hazard are suitable for the preparations according to the present invention.

“Alcoholic drinks” are ethanol-containing drinks, the ethanol content varying within 2.8% by volume and 84% by volume.

“Alcoholic foodstuffs” and “non-alcoholic beverages” refer to foodstuffs containing less than 2.8% of ethanol. Such foodstuffs and beverages can be, for example, fermented juices or preserves, or foodstuffs preserved with small amounts of alcohol, pastries, jellies, and mousse seasoned with liqueur or corresponding preparations containing alcohol.

The use of the preparations according to the invention can be of benefit even, when light alcoholic drinks or foodstuffs are consumed, which contain small amounts of alcohol. Some foodstuffs can also already contain acetaldehyde. Acetaldehyde-containing foodstuffs, which have ethanol that is generated in connection with fermentation, such as beer, cider, wine, home-brewed beer, and other alcoholic drinks, as well as many juices. Of the alcoholic drinks, grappa and sherry contain particularly high concentrations of acetaldehyde.

Thus, according to a preferred embodiment of the present invention, the preparation is administered to the subject in connection with eating, i.e., just before, during or just after eating, or in connection with consuming alcohol, i.e., just before, during or after consuming a dose of alcohol.

“In connection with consuming alcohol” herein refers to the period of time that begins from the onset of alcohol intake and ends, when no more alcohol is present in the blood.

Pharmaceutical Preparation Acting in the Stomach

“A long-acting preparation that has a local effect on the stomach” refers to all monolithic or multi-particular tablets or capsules or granules as such, which, when wetted under the influence of the gastric juices adhere to the mucous membrane of the stomach or form a gel that floats in the contents of the stomach, as a consequence of which their residence time in the stomach is prolonged and thus enables a prolonged release in and a local effect of the drug on the stomach.

A special property required of the pharmaceutical composition that has a local effect on the stomach is that it remains in the stomach for as long as possible. Technically, this can be solved in two ways: by making a preparation that adheres to the mucous membrane of the stomach or making a preparation that floats in the contents of the stomach. The preparation can be rendered fixable to the mucous membrane of the stomach by using as additives cationic polymers, such as various chitosan grades. Preparations that float in the stomach are provided by using polymers (e.g., alginic acid) that form a gel and by adding to the preparation sodium hydrogen carbonate, which under the influence of gastric acid releases carbon dioxide, which in turn forms gas bubbles inside the gel. A liquid gel that floats in the stomach can also be prepared from sodium alginate, aluminium hydroxide, sodium hydrogen carbonate, and water, to which the acetaldehyde-binding compound can be added. A corresponding liquid preparation is also obtained by adding an acetaldehyde-binding substance to an aqueous dispersion of chitosan.

Another preparation that remains in the stomach for a long time is a preparation, which is known as HBS™ (hydrodynamically balanced system). The preparation can remain in the stomach for a long time, when a relatively large tablet is made of it (with a diameter of at least 7-10 mm) and it is coated with a film, which does not decompose in the alimentary tract, and which, however, releases an effective substance (Oros™) through a hole which has been made to it, for example. However, a prerequisite is that such a preparation be consumed after eating.

A preferred single dose of the pharmaceutical composition having a local effect on the stomach comprises 50-500 mg of acetaldehyde-binding substance; preferably the amount of acetaldehyde-binding substance is 50-300 mg, and most preferably 100-200 mg. The amount of compound released in the conditions of the stomach is preferably 40-80 mg in an hour.

The preparation according to the invention, which releases in the stomach, has at least one—often two—polymers, which have the task of retaining the drug as long as possible, for two hours minimum, in the stomach either by attaching the preparation to the mucous membrane of the stomach or by forming a gel that floats in the contents of the stomach. Another function of the polymers is to prolong the release of the effective substance.

The preparation that locally binds acetaldehyde in the stomach can be a tablet that forms a gel in the stomach or a capsule comprising a mixture of powder or granules that forms a gel. In addition to the acetaldehyde-binding substances, the preparation comprises polymers that form a gel in the stomach, such as chitosans, alginates, sodium carboxy-methylcellulose grades, carbomers or aluminium hydroxide. To advance floating in the stomach, the preparation can also comprise sodium hydrogen carbonate.

The amount of polymers in the preparation is 10-50%, preferably 15-40%, and most preferably 20-30%.

The proportion of sodium hydrogen carbonate can be 10-30%, preferably 20% of the amount of polymers.

The preparation that locally binds acetaldehyde in the stomach can be a tablet or granule preparation, wherein the acetaldehyde-binding substance is mixed with the fillers needed and, after that, granulated by using enteric polymers as binders. The binder used can be any known enteric polymer, preferably a polymer with a solution pH of 6-7, and most preferably the polymer is any of the methacrylate derivatives, which are known by the trade names Eudragit L and Eudragit S. The amount of enteric polymer in the preparation is preferably 2-5%, most preferably 3-4%.

The preparation that locally binds acetaldehyde in the stomach can be a liquid preparation, i.e., a mixture comprising, in addition to the acetaldehyde-binding substance, also sodium alginate, aluminium hydroxide, sodium hydrogen carbonate, and water. The amount of water in the whole preparation is 70-90%, most preferably about 75-85%. The amount of sodium alginate in the preparation is preferably 2-10%, most preferably about 5%, and the amount of aluminium hydroxide is preferably 5-15%, most preferably about 10%.

The relative composition of the preparation comprising granules can be as follows, for example:

| | | |
|---|---|---|
| Acetaldehyde-binding substances | 60 | parts |
| Chitosan | 10-40 | parts |
| Calcium hydrogen phosphate | 0-30 | parts |

The relative composition of the liquid preparation can be as follows, for example:

| | | |
|---|---|---|
| Acetaldehyde-binding substances | 10 | parts |
| Sodium alginate | 2-10 | parts |
| Aluminium hydroxide | 5-15 | parts |
| Sodium hydrogen carbonate | 1-2 | parts |
| Water | 70-80 | parts |

Preparations for Binding Aldehydes in the Mouth

One embodiment of the invention is to use a preparation that is to be placed in the mouth of a subject and contains a combination of active compounds including one or more cysteine compounds (i.e. aldehyde-binding compounds) that are intended for binding the aldehydes, such as the acetaldehyde, formaldehyde, acrylic aldehyde, propionaldehyde and butyraldehyde, in the mouth, and xylitol intended for destroying (i.e. killing) at least some of the microbes present in the mouth, as well as one or more non-toxic additives, which are harmless for human (or animal) consumption. Furthermore, xylitol has been shown to inhibit microbial acetaldehyde formation from ethanol (Uittamo et al. 2011).

The composition functions by the cysteine compound(s) binding at least some of the aldehydes into a harmless form, whereas the xylitol kills or inhibits at least some of the microbes in the mouth that are responsible for generating aldehydes. Thus, the effect is synergistic, as a smaller amount of aldehydes will be produced, and an effective binding of said smaller amount of aldehydes is achieved, thus resulting in a considerably more efficient reduction of the aldehyde contents in the saliva compared to the contents achieved using prior solutions.

One objective of using said additive(s) is to bind the active compounds into a lozenge, or into a buccal or sublingual tablet. Particularly, at least one of the additives is selected from the carriers or binders capable of causing the sustained release of said cysteine compound(s) from the lozenge or the tablet into the saliva in the conditions of the mouth.

According to a preferred embodiment, at least one of the additives of the composition is formed into a coating on the lozenge or the tablet containing the remaining constituents of the composition. Preferably, such a coating is formed from at least a portion of the xylitol or at least a portion of any further aromatic agents of the composition.

The term “additive” here includes carriers, fillers and binders, as well as aromatic agents colorants and non-functional additives. These additives are non-toxic, and preferably control the release of the active agents to take place specifically in the mouth, and most suitably in a sustained manner. These formulations are intended to be placed in the mouth, for example between the cheek or the lip and the gum, or they are intended to be sucked.

The composition comprises an effective amount of the above mentioned cysteine compound(s). Here an effective amount means an amount capable of binding or inactivating the amount of aldehyde carried to the mouth from foodstuff, drinks or tobacco, or formed in the mouth, for example by the microbial activity therein, during the digestion of foodstuff or drinks in connection with or after the consumption.

Typically, a single unit, or formulation, of the composition comprises 1-30 mg, preferably 1-20 mg, more preferably 1-10 mg, and most suitably 2-6 mg of the cysteine(s). However, 1-2 of these units can be administered at once.

The content of the cysteine compound(s) is then preferably 1-50%, more preferably 5-40%, most suitably 20-30%, of the weight of the composition. Typically, the content is 20-25 w-%.

The composition also comprises an effective amount of the above mentioned xylitol. Here an effective amount means an amount capable of at least causing a measurable inactivation of the acetaldehyde-producing bacteria in the mouth or significantly inhibiting microbial acetaldehyde formation from alcohol.

Typically, a single unit, or formulation, of the composition comprises 50-500 mg, preferably 50-300 mg, more preferably 100-300 mg, and most suitably 200-300 mg of xylitol. However, as stated above, 1-2 units can be administered at once.

The content of xylitol is then preferably 10-90%, more preferably 10-60%, particularly 20-60%, and most suitably 40-60% of the weight of the composition. Typically, the content is about 50%.

A lozenge according to the present invention may e.g. comprise:

| | | | |
|---|---|---|---|
| L-Cysteine | 3 | mg | (0.6%) |
| Sorbitol | 224 | mg | (44.8%) |
| Xylitol | 250 | mg | (50.0%) |
| Aromatic (flavouring) agent(s) | 12.5 | mg | (2.5%) |
| Magnesium stearate | 10 | mg | (2.0%) |
| Silicon dioxide | 0.5 | mg | (0.1%) |

A sucking tablet according to the present invention may e.g. comprise:

| L-cysteine | 20 | mg |
|---|---|---|
| Xylitol (or an equivalent sugar or sugar alcohol) | 750 | mg |
| Flavouring | q.s. | |
| Magnesium stearate | 10 | mg |

A sublingual tablet according to the present invention may e.g. comprise:

| L-cysteine | 10 | mg |
|---|---|---|
| Xylitol | 250 | mg |
| Flavouring | q.s. | |
| Magnesium stearate | 5 | mg |

Pharmaceutical Preparation Acting in the Large Intestine

“A long-acting preparation that has a local effect in the large intestine” refers to all monolithic or multi-particular tablets or capsules or granules as such, which will not release the dose in a prolonged way until the preparation has drifted to the end of the small intestines or all the way to the large intestine.

The preparation according to the invention that releases acetaldehyde-binding substances in the large intestine in a prolonged way, carries the acetaldehyde-binding substance to the last part of the small intestine or to the large intestine before the substance in question is allowed to be released—whichever the releasing mechanism.

The pharmaceutical composition that binds acetaldehyde in the large intestine is administered orally. There are different techniques available for directing the release of an orally dosed drug to the large intestine. The most functional solutions are based on the use of enteric polymers. A film coating, which does not dissolve in the acidic environment of the stomach, but dissolves at pH 7 at the latest, can be made both on the tablet and the granules. In making the preparation, it is also possible to use polysaccharides that degrade under the effect of microbes of the large intestine, or polymers generated by azo bonds. The form of preparation known by the trade name Oros™ can also be used, when its opening is first covered with an enteric polymer, the solution pH of which is ≈7.

Useful enteric polymers include, for example, the grades of hydroxypropyl methylcellulose-acetatesuccinate (HPMC-AS) sold by the trade name Aqoat™, Aqoat AS-HF™ in particular, a cellulose acetatephtalate (CAP) grade sold by the trade name Aquateric™, and methacrylic acid-methylmethacrylate copolymers, the grade sold by the trade name Eudragit-S™ in particular.

The preparation according to the invention has at least one ingredient, which adjusts the release of the effective substance not to take place until at the end of the small intestine or in the large intestine. This component can be a polymer that dissolves depending on the pH (=enteric polymer) or a polymer that degrades under the effect of the enzymes secreted by the bacteria of the large intestine. The polymer that controls the place of release can form a film around the entire preparation. It can also form a film around the particles (granules) contained by the multiple-part preparation. The polymer that degrades under the effect of the enzymes secreted by the bacteria of the large intestine can also be as a filler in a monolithic preparation, or as a filler in the granules or in a multiple-unit preparation prepared from these granules.

The preparation according to the invention is an enteric tablet, the film coating of which does not dissolve until at the end of the small intestine or at the beginning of the large intestine. The dissolution pH of the polymer that forms the film is 6.0-7.5, preferably 6.5-7.0. The amount of enteric polymer that forms the film is 5-20%, preferably 10-15% of the whole mass of the tablet. The filler of the tablet can comprise pharmaceutical additives that do not swell, such as calcium hydrogen phosphate.

The preparation according to the invention can also be granules that comprise an acetaldehyde-binding substance and are coated with an enteric film, the dissolution pH of the film-forming polymer being 6.0-7.5, preferably 6.5-7.0. The amount of film-forming enteric polymer of the entire mass of the granule is 5-30%, preferably 15-25%. The granule can comprise 20-40%, preferably about 30% of filler poorly soluble in water, such as calcium hydrogen phosphate.

The binder of the granule coated with the enteric film, according to the invention, can be an enteric polymer, the dissolution pH of which is 6.0-7.5, preferably 6.5-7.0. The amount of binder in the granule is 2-5%, preferably 3-4%.

The preparation according to the invention can also be a tablet comprising the enteric coated granules described above, on which an enteric film has also been made. The tablet made for such a preparation not only comprises enteric granules, but also a filler suitable for direct compression, such as microcrystalline cellulose, the amount of which in the tablet is 30-70%, preferably 40-60%.

The dosage unit of the pharmaceutical composition preferably comprises 50-500 mg of acetaldehyde-binding substance; preferably the amount of acetaldehyde-binding substance is 50-300 mg, and most preferably 100-200 mg.

The amount of compound releasing in the conditions of the large intestine is preferably 50-100 mg in an hour.

The composition of the enteric tablet, which comprises enteric granules and binds acetaldehyde in the desired way, can be as follows, for example:

Enteric granules:

| | | |
|---|---|---|
| Acetaldehyde-binding substance | 100 | mg |
| Filler, e.g., calcium hydrogen phosphate | 30-50 | mg |
| Enteric polymers | 40-60 | mg |

Enteric tablet:

| | | |
|---|---|---|
| Enteric granules | 170-210 | mg |
| Microcrystalline cellulose | 170-210 | mg |
| Lubricants (e.g. magnesium stearate and talcum) | 5-10 | mg |
| Enteric polymers | 30-50 | mg |

Slowly L-Cysteine Releasing Preparations in the Elimination of Acetaldehyde

As evident from the present disclosure, cysteine, a semi-essential amino acid, is able to eliminate the toxicity of acetaldehyde by binding to it covalently. The inactive product is stable 2-methylthiazolidine-4-carboxylic acid (MTCA).

On the basis of the present invention, slowly L-cysteine releasing preparations can be used to eliminate the histamine-releasing effect of locally formed acetaldehyde in the digestive tract. This concept is supported by the following findings.

Orally administered slowly L-cysteine releasing preparations have been successfully used to eliminate free and active acetaldehyde in saliva during alcohol consumption and smoking (Salaspuro et al. 2002, 2006, Kartal et al. 2007). Furthermore, slowly L-cysteine releasing capsules (Acetium® capsules) have been shown to eliminate effectively acetaldehyde in gastric juice of patients with atrophic gastritis after alcohol administration (Linderborg et al. 2011, Hellström et al. 2014).

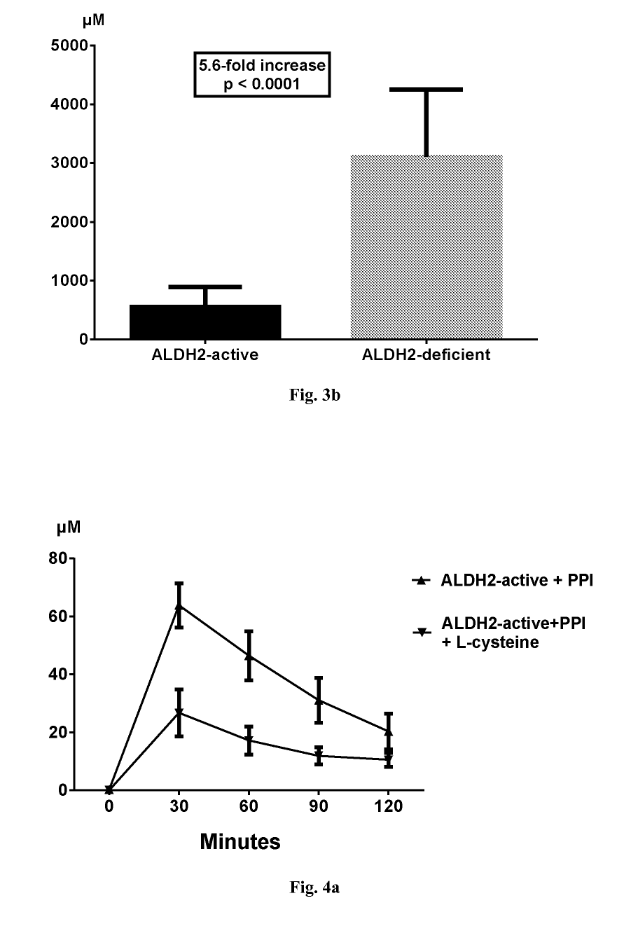

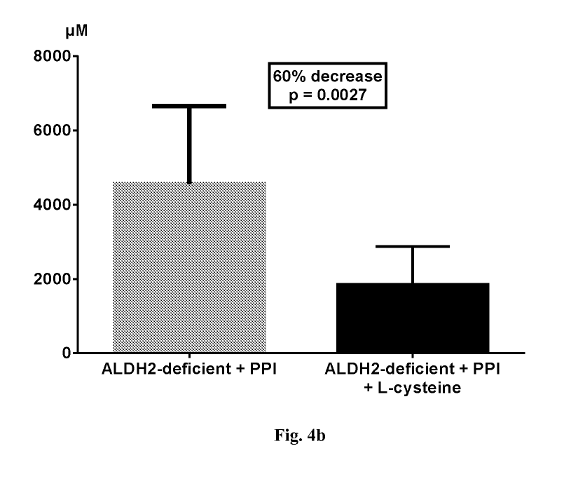

Most recently, it was found that after intragastric infusion of ethanol, gastric juice acetaldehyde levels in flushing ALDH2-deficient Japanese subjects are 5.6-fold higher as compared to those who have normal ALDH2-enzyme (FIGS. 3a and 3b). Most importantly, 60% of gastric juice acetaldehyde could be eliminated by slowly L-cysteine releasing Acetium® capsules (FIGS. 4a and 4b). The effect lasted for 2 hours.

Thereby, one embodiment of the present invention is a cysteine-releasing pharmaceutical preparation, such as slowly L-cysteine releasing pharmaceutical preparation, for use in preventing and/or treating alcohol flushing and alcohol-induced hypersensitivity reactions. In this context, alcohol flushing is typically characterized by facial redness, increased skin temperature and heart rate and decreased diastolic blood pressure, and the alcohol induced hypersensitivity reactions are preferably selected from allergic rhinitis (hay fever), allergic conjunctivitis, atopic dermatitis, eosinophilic esophagitis, anaphylaxis, chronic bronchitis and chronic obstructive pulmonary disease (COPD).

According to another embodiment of the invention, the cysteine preferably comprises L-cysteine, D-cysteine, N-acetyl cysteine or any other derivative of cysteine and any pharmaceutically acceptable salts thereof.

According to one embodiment of the invention, the pharmaceutical preparation comprises a cysteine both in the inner structure(s) and in the tablet material surrounding the inner structure(s).

According to a further embodiment of the present invention, the pharmaceutical preparation is in the form of a capsule. In such case a dosage regime of 1-2 slowly L-cysteine releasing capsules containing 5-200 mg L-cysteine taken 20 minutes before and/or immediately after intake of a dose of alcohol and repeating such dose at 1-2 hours intervals for as long as alcohol drinking continues is preferably used. However, another equally good dosage regime is to use 1-2 slowly L-cysteine releasing capsules containing 5-200 mg L-cysteine taken 20 minutes before and/or immediately after intake of any food or beverage if there is any suspicion that the food or beverage contains any alcohol and/or acetaldehyde is used.

According to even further embodiment of the invention, a dosage regime of 100 mg-2 g of L-cysteine (without slow release) given orally before and/or immediately after intake of any food or beverage if there is any suspicion that the food or beverage contains any alcohol and/or acetaldehyde is used. Thus, according to this one embodiment, high cysteine dose results in the same desired effect as the slowly cysteine releasing preparation, which is being administered as lower dose.

The cysteine releasing pharmaceutical preparation may also be in the form of a lozenge or a chewing gum or any other similar preparation, which is used either alone or together with cysteine-releasing capsules and following the previously described dosage regimes.

One further suitable dosage regime comprise 1 slowly L-cysteine releasing lozenge, chewing gum or any similar preparation containing 2-6 mg, preferably 2-3 mg L-cysteine taken just before the intake of a dose of alcohol or any beverage or food suspected to contain any alcohol and/or acetaldehyde. However, it may be beneficial to take another slowly L-cysteine releasing, lozenge, chewing gum or any similar preparation containing 2-6 mg, preferably 2-3 mg L-cysteine just after a dose of alcohol or any beverage or food suspected to contain any alcohol and/or acetaldehyde.

According to one embodiment the preparations for use according to the present invention include for example the commercial preparations under trademark Acetium® (both capsule and lozenge, Biohit Oyj, Finland).

As disclosed earlier in the present application, a method for eliminating histamine-releasing effect of locally formed acetaldehyde in the digestive tract is covered by the present invention. Such method comprises introducing an acetaldehyde-binding substance into the digestive tract by swallowing, sucking or chewing slowly L-cysteine releasing preparation(s) and thus permitting L-cysteine to bind with acetaldehyde and inactivate it.

The method preferably includes the steps of:
swallowing 1-2 slowly L-cysteine releasing capsules containing 50-200 L-cysteine 20 minutes before drinking alcohol, and
repeating such dose at 1-2-hour intervals for as long as alcohol drinking continues.

In another embodiment the method includes the step(s) of:
sucking or chewing 1 slowly L-cysteine releasing lozenge, chewing gum or any similar preparation containing 2-3 mg L-cysteine just before the intake of a dose of alcohol.
another L-cysteine releasing lozenge or chewing gum is taken immediately after a shot of alcoholic beverage or after eating or drinking any alcohol and/or acetaldehyde containing foodstuffs or beverages.

It should be understood by the skilled reader, however, that the embodiments given in the description above are for illustrative purposes only, and that various changes and modifications are possible within the scope of the claims hereto.

### REFERENCES AND RELATED PUBLICATIONS

IARC monographs on the evaluation of carcinogenic risks to humans. Personal habits and indoor combustions, volume 100E (2012).

Salaspuro M P, Acetaldehyde, microbes and cancer of the digestive tract. Crit Rev Clin Lab Sci 2003; 40:183-208.

Salaspuro M. Acetaldehyde as a common denominator and cumulative carcinogen in digestive tract cancers. Scand J Gastroenterol 2009; 44:912-25.

Salaspuro M. Acetaldehyde and gastric cancer. J Dig Dis 2011; 12:51-9.

Kupari M, Eriksson C J, Heikkilä J, Ylikahri R. Alcohol and the heart. Intense hemodynamic changes associating with alcohol flush in Orientals. Acta Med Scand 1983; 213:91-98.

Väkeväinen S, Tillonen J, Salaspuro M. 4-methylpyrazole decreases salivary acetaldehyde levels in ALDH2-deficient subjects but not in subjects with normal ALDH2. Alcohol Clin Exp Res 2001; 25:829-34.

Brooks P J, Enoch M-A, Goldman D, Yokoyama A. The alcohol flushing response: An unrecognized risk factor for esophageal cancer from alcohol consumption. PLoS Medicine 2009; 6:258-63.

Shimoda T, Kohno S, Takao A et al. Investigation of the mechanism of alcohol-induced bronchial asthma. J Allergy Clin Immunol 1996; 97:74-84.

Koivisto T, Kaihovaara P, Salaspuro M. Acetaldehyde induces histamine release from purified rat peritoneal mast cells. Life Sci 1999; 64:183-90.

Nihlen U, Greiff L J, Nyberg P. et al. Alcohol-induced upper airway symptoms: prevalence and comorbidity. Respir Med 2005; 99:726-9.

Linneberg A, Berg N D, Gonzales-Quintela. A et al. Prevalence of self-reported hypersensitivity symptoms following intake of alcoholic drinks. Clin Exp Allergy 2008; 38:145-51.

Miller N S, Goodwin D W, Jones F C et al. Antihistamine blockade of alcohol-induced flushing in Orientals. J Stud Alcohol 1988; 49;16-20.

Myou S, Fujimura M, Nishi K et al. Inhibitory effect of terfenadine, a selective H1 histamine antagonist, on alcoholic beverage-induced bronchoconstriction in asthmatic patients. Eur Respir J 1995; 8:619-23.

Takao A, Shimoda T, Matsuse H et al. Inhibitory effects of azelastine hydrochloride in alcohol-induced asthma. Ann Allergy Asthma Immunol 1999; 82:390-4.

Linneberg A, Gonzales-Quintela A, Vidal C et al. Genetic determinants of both ethanol and acetaldehyde metabolism influence alcohol hypersensitivity and drinking behavior among Scandinavians. Clin Exp Allergy 2010; 40:123-30.

Lindros K O, Stowell A, Pikkarainen P, Salaspuro M. Elevated blood acetaldehyde in alcoholics with accelerated ethanol elimination. Pharmacol Biochem Behav 1980; 13 Suppl 1:119-24.

Homann N, Jousimies-Somer H, Jokelainen K, Heine R, Salaspuro M. High acetaldehyde levels in saliva after ethanol consumption: Methodological aspects and pathogenetic implications. Carcinogenesis 1997; 18:1739-43.

Väkeväinen S, Tillonen J, Agarwal D P, Srivastava N, Salaspuro M. High salivary acetaldehyde after a moderate dose of alcohol in ALDH2-deficient subjects: Strong evidence for the local carcinogenic action of acetaldehyde. Alcohol Clin Exp Med 2000; 24:873-77.

Dong Y J, Peng T K, Yin S J. Expression and activities of class IV alcohol dehydrogenase and class III aldehyde dehydrogenase in human mouth. Alcohol 1996; 13:257-62.

Mizoi Y, Iijiri I, Tatsuno Y et al. Relationship between facial flushing and blood acetaldehyde levels after alcohol intake. Pharmacol Biochem Behav 1979; 10:303-11.

Chen Y-C, Lu R-B, Peng G-S et al. Alcohol metabolism and cardiovascular response in alcoholic patient homozygous for the ALDH2*2 variant gene allele. Alcohol Clin Exp Res 1999; 23:1853-60.

Yokoyarna A, Tsutsumi E, Imazeki H et al. Salivary acetaldehyde concentration according to alcoholic beverage consumed and aldehyde dehydrogenase-2 genotype. Alcohol Clin Exp Res 2008; 32;1607-14.

Steer H W. Mast cells of the human stomach. J Anat 1976; 121:385-97.

Syrjänen K J. Morphologic manifestations of tumor-host relationships in association with breast, gastric and colorectal carcinoma. M.D. (Ph.D.) Thesis, University of Helsinki, pp. 1-86, 1975. (ISBN-951-99064-3-6).

Gomes A P, Johann J E, Lovato G G, Ferreira A M. Comparative analysis of the mast cell density in normal oral mucosa, actinic cheilitis and lip squamous cell carcinoma. Braz Dent J 2008; 19:186-9.

Sprince H, Parker C M, Smith G G, Gonzales L J. Protective action of ascorbic acid and sulfur compounds against acetaldehyde toxicity: implications in alcoholism and smoking. Agents Actions 1976; 5164-73.

Salaspuro V, Hietala J, Kaihovaara P, Pihlajarinne L, Marvola M, Salaspuro M. Removal of acetaldehyde from saliva by slow-release buccal tablet of L-cysteine. Int J Cancer 2002; 97:361-4.

Salaspuro V J, Hietala J M, Marvola M L, Salaspuro M P. Eliminating carcinogenic acetaldehyde by cysteine from saliva during smoking. Cancer Epid Biomark Prev 2006; 15:146-49.

Kartal A, Hietala J, Laakso I, Kaihovaara P, Salaspuro V, Säkkinen M, Salaspuro M, Marvola M. Formulating and in-vivo evaluation of L-cysteine chewing gums for binding carcinogenic acetaldehyde in saliva during smoking. J Pharm Pharmacol 2007; 59:1353-8.

Linderborg K, Marvola T, Marvola M, Salaspuro M, Färkkilä M, Väkeväinen S. Reducing carcinogenic acetaldehyde exposure in the achlorhydric stomach with cysteine. Alcohol Clin Exp Res 2011; 35:516-22.

Hellström P M, Hendolin P H, Kaihovaara P et al. L-cysteine slow-release capsule formulation in prevention of gastric carcinogenesis associated with atrophic gastritis. Gastroenterology 2014; 146 Suppl. 1, S-315, Sa1863.

Uittamo J, Nieminen M T, Kaihovaara P, Bowyer P, Salaspuro M, Rautemaa R. Xylitol onhibits carcinogenic acetaldehyde production by Candida species. Int J Cancer 2011; 129:2038-41.

### PATENT LITERATURE

WO 2012/027603

WO 2014/011676

US 2014/256760

1. A slowly cysteine-releasing pharmaceutical preparation for use in preventing and treating alcohol flushing and alcohol-induced hypersensitivity reactions by eliminating histamine-releasing effect of locally formed acetaldehyde in the digestive tract.

2. The preparation according to claim 1 for use in preventing and treating alcohol flushing and alcohol-induced hypersensitivity reactions, wherein alcohol flushing is characterized by facial redness, increased skin temperature and heart rate and decreased diastolic blood pressure.

3. The preparation according to claim 1 for use in preventing and treating alcohol flushing and alcohol-induced hypersensitivity reactions, characterized in that the alcohol-induced hypersensitivity reactions are selected from allergic rhinitis (hay fever), allergic conjunctivitis, atopic dermatitis, eosinophilic esophagitis, anaphylaxis, chronic bronchitis and chronic obstructive pulmonary disease (COPD).

4. The preparation according to claim 1 for use in preventing and treating alcohol flushing and alcohol-induced hypersensitivity reactions, wherein cysteine comprises L-cysteine, D-cysteine, N-acetyl cysteine or any other derivative of cysteine and any pharmaceutically acceptable salt thereof.

5. The preparation according to claim 1 for use in preventing and treating alcohol flushing and alcohol-induced hypersensitivity reactions, wherein the preparation comprises cysteine both in the inner structure(s) and in the tablet material surrounding the inner structure(s).

6. The preparation according to claim 1 for use in preventing and treating alcohol flushing and alcohol-induced hypersensitivity reactions, characterized in that the preparation is in the form of a capsule.

7. The preparation according to claim 6 for use in preventing and treating alcohol flushing and alcohol-induced hypersensitivity reactions, characterized in that a dosage regime of 1-2 slowly L-cysteine releasing capsules containing 50-200 mg L-cysteine taken 20 minutes before or immediately after intake of a dose of alcohol and repeating such dose at 1-2 hours intervals for as long as alcohol drinking continues is used.

8. The preparation according to claim 6 for use in preventing and treating alcohol flushing and alcohol-induced hypersensitivity reactions, characterized in that a dosage regime of 1-2 slowly L-cysteine releasing capsules containing 50-200 mg L-cysteine taken 20 minutes before or immediately after intake of any food or beverage if there is any suspicion that the food or beverage contains any alcohol and/or acetaldehyde is used.

9. The preparation according to claim 6 for use in preventing and treating alcohol flushing and alcohol-induced hypersensitivy actions, characterized in that a dosage regime of 100 mg-2 g of L-cysteine without slow release effect given orally before or immediately after intake of any food or beverage if there is any suspicion that the food or beverage contains any alcohol and/or acetaldehyde is used.

10. The preparation according to claim 1 for use in preventing and treating alcohol flushing and alcohol-induced hypersensitivity reactions, characterized in that the preparation is in a form of a lozenge, a chewing gum or any other similar preparation and is used either alone or together with cysteine releasing capsules and in the latter case following the dosage regime of 1-2 slowly L-cysteine releasing capsules containing 50-200 mg L-cysteine taken 20 minutes before or immediately after intake of a dose of alcohol and repeating such dose at 1-2 hours intervals for as long as alcohol drinking continues is used.

11. The preparation according to claim 10 for use in preventing and treating alcohol flushing and alcohol-induced hypersensitivity reactions, characterized in that a dosage regime of 1 slowly L-cysteine releasing lozenge, chewing gum or any similar preparation containing 2-3 mg L-cysteine taken just before the intake of a dose of alcohol or any beverage or food suspected to contain any alcohol and/or acetaldehyde is used.

12. The preparation according to claim 10 for use in preventing and treating alcohol flushing and alcohol-induced hypersensitivity reactions, characterized in that another slowly L-cysteine releasing, lozenge, chewing gum or any similar preparation containing 2-3 mg L-cysteine is taken just after a dose of alcohol or food or beverage suspected to contain any alcohol and/or acetaldehyde.

13. Use of a cysteine-releasing pharmaceutical preparation in preventing alcohol flushing and alcohol-induced hypersensitivity reactions, selected from allergic rhinitis (hay fever), allergic conjunctivitis, atopic dermatitis, eosinophilic esophagitis, anaphylaxis, chronic bronchitis and chronic obstructive pulmonary disease (COPD).

14. A method for preventing histamine-releasing effect of locally formed acetaldehyde in the digestive tract, wherein the method an acetaldehyde-binding substance is introduced into the digestive tract by swallowing, sucking or chewing slowly L-cysteine releasing preparation(s) and thereby permitting the L-cysteine to bind with acetaldehyde and inactivate it.

15. The method of claim 14, characterized in swallowing 1-2 slowly L-cysteine releasing capsules containing 50-200 mg L-cysteine 20 minutes before drinking alcohol and repeating such dose at 1-2 hours intervals for as long as alcohol drinking continues.

16. The method according to claim 14, characterized in sucking or chewing slowly L-cysteine releasing lozenge, chewing gum or any similar preparation containing 2-3 mg L-cysteine just before the intake of a dose of alcohol.
